(19) United States
(12) Patent Application Publication
(10) Pub. No.: US 2018/0065874 A1
(43) Pub. Date: Mar. 8, 2018

(54) REDUCTION OF DISINFECTION BYPRODUCT FORMATION IN DRINKING WATER

(71) Applicant: PeroxyChem LLC, Philadelphia, PA (US)

(72) Inventors: Kwok-Keung AU, Naperville, IL (US); Alberto GARIBI, Haddonfield, NJ (US); Philip BLOCK, Garnet Valley, PA (US)

(73) Assignee: PeroxyChem LLC, Philadelphia, PA (US)

(21) Appl. No.: 15/691,101

(22) Filed: Aug. 30, 2017

Related U.S. Application Data
(60) Provisional application No. 62/383,009, filed on Sep. 2, 2016.

(51) Int. Cl. C02F 1/72; C02F 1/76; C07C 53/00

(52) U.S. Cl. CPC C02F 1/722; C02F 2101/34; C07C 53/00; C02F 1/76

(57) ABSTRACT

Disclosed herein are methods and compositions for the reduction of disinfection byproduct precursors in raw drinking water.

### CROSS REFERENCE TO RELATED APPLICATIONS

This application claims the benefit of the filing date of U.S. Provisional Application No. 62/383,009 which was filed Sep. 2, 2016. The entire content of U.S. Provisional Application No. 62/383,009 is hereby incorporated by reference herein.

### FIELD OF THE INVENTION

The present invention relates to the reduction of disinfection byproduct formation in drinking waters through the oxidation of precursors by peracetic acid prior to the disinfection process.

### BACKGROUND OF THE INVENTION

Chlorination is a technology used in the disinfection of drinking water. Chlorination has significantly reduced the incidence of human disease and is one of the most significant contributions to the improvement of human health over the past century. The ability of chlorine to provide a stable residual concentration makes it suitable as a drinking water disinfectant at the point of use. However, natural organic matter in the raw water being treated and disinfected at the drinking water treatment plant may interact with residual chlorine to form compounds classified as disinfection by-products. The most commonly employed disinfectants include chlorine, chloramines, chlorine dioxide and ozone, each of which can generate a variety of disinfection by-products. Such disinfection by-products may include various halogenated species, including trihalomethanes and haloacetic acids. Exemplary trihalomethanes generated during the chlorination of drinking water include: chloroform, dibromochloromethane, bromoform, bromodichloromethane and similar species. Exemplary haloacetic acids formed during chlorine disinfection include: trichloracetic acid, tribromoacetic acid, monochloroacetic acid, monobromoacetic acid, dichloroacetic acid, dribromoacetic acid, chlorodibromoacetic acid, broodichloracetic acid and bromochloracetic acid.

Disinfection by-products are recognized as potentially carcinogenic and many are reported to be cytotoxic, neurotoxic, mutagenic, or teratogenetic (Plewa et al. Environ. Sci. Technol., 2008, 42 (3), pp 955-961). The United States Environmental Protection Agency has instituted controls to reduce and eliminate disinfection by-products from drinking water by setting a maximum allowable limited on trihalomethanes. Federal Code 40 CFR Parts 9, 41 and 142, sets the national primary drinking water regulations for disinfection by-products and sets maximum limits on trihalomethanes and haloacetic acids. Several methods are outlined in the Federal Code with regards to reducing the formation of disinfection by-products, including limiting the maximum residual concentration of chlorine and chlorine based disinfectants, removal of total organic carbon, and enhanced coagulation.

West et al. (Chemosphere (2016) 153:21-527) discloses that replacement of free chlorine or chloramines with peracetic acid as the primary disinfectant can reduce the potential formation of N-nitrosamines. WO2007087345A3 discloses that addition of a combination of peroxynitrite and another oxidant, such as peracetic acid, can be used to oxidize contaminants in wastewater.

Peracetic acid has been utilized for the disinfection of medical devices, hard surfaces, carcasses and more recently as a disinfection technology for municipal and industrial wastewaters. To date, it has not been utilized as the final disinfecting agent in drinking water applications due, in part, to its relatively shorter term residual concentration compared to chlorine-based technologies.

### SUMMARY OF THE INVENTION

The present invention relates to a method of treating raw drinking water containing natural organic matter or other disinfection by-product precursors. The method of the present invention uses peracid solution prior to the addition of disinfection by-product-forming disinfection chemicals under conditions that substantially reduce or prevent the formation of trihalomethanes (THM) and haloacetic acid (HAA) disinfection by-products in the final, treated drinking water. Peracetic acid, performic acid and perpropianic acid may be used for this purpose as well, or in combination with each other.

The target concentration of the peracid can be controlled via a number of different control schemes. Flow pace control utilizes controlling the flow rate of the peracid into the raw water stream by scaling the flow to the measured flow rate rate of the raw water stream. Feed-back residual control utilizes the signal output of an ampeometric, submersible, peracid analytical probe to adjust the peracid flow rate to maintain a target peracacid concentration in the raw water stream. Feed-forward demand control measures the total organic carbon content or the chemical oxidant demand of the raw water stream and adjusts the peracid flow rate to achieve the desired peracid concentration in the raw water stream that is need to oxidize most, and preferably substantially all of the total organic carbon or chemical oxidant demand. In some embodiments, one or more of the three control schemes listed above can be combined.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 displays the results of adding peracetic acid at various concentrations to untreated raw water on the prevention of forming trihalomethanes and haloacetic acids.

### DETAILED DESCRIPTION OF THE PREFERRED EMBODIMENT

This description of preferred embodiments is intended to be read in connection with the accompanying drawings, which are to be considered part of the entire written description of this invention. The drawing FIGURES are not necessarily to scale and certain features of the invention may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing FIGURE under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The term “operatively connected” is such an attachment, coupling or connection that allows the pertinent structures to operate as intended by virtue of that relationship. When only a single machine is illustrated, the term “machine” shall also be taken to include any collection of machines that individually or jointly execute a set (or multiple sets) of instructions to perform any one or more of the methodologies discussed herein. In the claims, means-plus-function clauses, if used, are intended to cover the structures described, suggested, or rendered obvious by the written description or drawings for performing the recited function, including not only structural equivalents but also equivalent structures.

The methods disclosed herein are generally useful for the reduction in the levels of disinfection byproduct precursors in a water sample, for example, raw drinking water. The methods relate to oxidation of disinfection by-products precursors by a peracid, such as peracetic acid. Treatment of raw drinking water with peracetic acid before the water is exposed to chlorine disinfection reduces the level of disinfection byproduct precursors. Subsequent exposure of the peracetic acid treated water to chlorine disinfection produces a drinking water effluent with substantially reduced levels of disinfection byproducts.

A disinfection byproduct precursor can be, for example, natural organic matter such as humus. In some embodiments, a disinfection byproduct precursor can be fulvic acid or humic acids, or amino acids.

In accordance with the process of the present invention, disinfection by-products, including trihalomethanes and haloacetic acids, are reduced in the final disinfected drinking water by contacting the raw water containing natural organic matter or disinfection by-product precursors with peracid prior to the application of disinfection chemicals. A raw water sample can be water that has not been contacted with a disinfection chemical. The peracetic acid is used to treat “raw water” entering, for example, a drinking water purification facility. The peracetic acid is added to the water treatment process in a “pre-oxidation” or “pre-disinfection” stage. The peracetic acid oxidizes raw water components, for example, organic materials such as humic acid or fulvic acid, that would otherwise be converted into trihalomethanes and haloacetic acids upon exposure to typical chlorine-based disinfectant. Such pre-treatment can be carried out under conditions which assures that most, or substantially all of the trihalomethanes and haloacetic acids are eliminated from the final, drinking water effluent.

In some embodiments, the peracid solution is a peracetic acid solution. The peracetic acid solution can be added to the raw water in drinking water treatment process prior in a “pre-oxidation” or “pre-disinfection” stage in concentrations of 0.5 to 20 mg peracetic acid per liter of water.

Peracetic acid solutions exist as equilibrium solutions containing peracetic acid, hydrogen peroxide, acetic acid and water. Solutions are often identified by the concentration of peracetic acid and hydrogen peroxide. For example, a 15/23 formulation contains 15% by weight of peracetic acid and 23% by weight hydrogen peroxide. Commercially available peracetic acid solutions have typical formulations containing 2-35% peracetic acid and 5-30% hydrogen peroxide, with the remainder being acetic acid and water.

The concentration of the peracetic acid in the peracetic acid solution used to achieve the target concentrations in the raw water can vary. Useful concentrations range from 2 to 35% by weight. In some embodiments the peracetic acid solution contains peracetic acid in the concentration range of 15 to 22 percent by weight.

The peracetic acid concentration in the raw water can be controlled by a flow-pacing scheme in which the peracetic acid solution addition rate is scaled to the flow rate of the raw water stream. In some embodiments, the peracetic acid solution addition rate is controlled via a feed-back signal from a peracetic acid, analytical, submersible probe to achieve a specific target concentration of peracetic acid in the raw water. In some embodiments, the total organic content or the chemical oxygen demand of the raw water is measured and used to control the peracetic acid solution addition rate. Alternatively, one or more of the control schemes listed above can be combined.

The peracetic acid treated water can then be contacted with one or more disinfecting chemicals. The specific disinfecting chemicals can vary. Exemplary disinfecting chemicals include chlorine, chloramines, chlorine dioxide, permaganate, and ozone.

### EXAMPLES

### Example 1

A sample of untreated, raw water was obtained from an undisclosed location in Texas and was received within one day from the time the sample was collected. The sample was refrigerated overnight, and testing was performed the following day after receipt.

Two liter aliquots of the sample were placed in four different cleaned and disinfected beakers and set on a Phipps and Bird jar tester apparatus. The stirrers were set to 100 rpm for the duration of the test.

A peracetic acid solution containing 15% by weight of peractic acid and 23% by weight of hydrogen peroxide was utilized for this test.

The peracetic acid solution was added three of the beakers to achieve initial peracetic acid concentrations of 1, 5 and 10 mg peracetic acid/L of raw water, respectively. The fourth beaker did not receive peracetic acid and served as a control. The peracetic acid concentration was initially measured twenty to thirty seconds after addition of the peracetic acid to the beaker. After sixty minutes, a stoichiometric amount of a sodium thio sulfate was added to each beaker containing peracetic acid in order to quench the peracetic acid and prevent further reaction, and the jars were stirred for an additional five minutes in order to provide sufficient time for neutralization.

The neutralized samples and the control were then packed in sampling containers and shipped to a third party laboratory for analytical testing.

Analytical testing included measurement of THM Formation Potential (THMFP) and the HAA Formation Potential (HAAFP). The THMFP and HAAFP were peformed via the standard test method 5710B, and detection of THM and HAA were measured by standard test method 524.2 and 552.2 respectively. In brief, the quenched samples were exposed to chlorine, which would result in the formation of THM or HAA if the quenched samples contained disinfection byproduct precursors that could potentially form THM or HAA. All methods were based on those described in Standard Methods for the Examination of Water and Wastewater, edited by E. W. Rice, R. B. Baird, A. D. Eaton, and L. S. Clesceri, co-published by American Public Health Association, Water Environment Federation, and American Water Works Association.

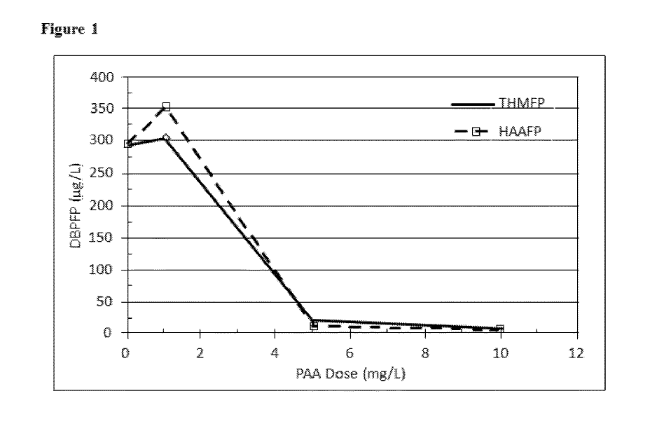

The reduction in THM Formation Potential and HAA Formation Potential after 60 minutes of contact as a function of peracetic acid concentration is shown in Table 1. These data are also presented graphically in FIG. 1.

TABLE 1
THMFP and HAAFP at various PAA Doses

| Peracetic acid (mg/L) | THMFP (μ/L) | HAAFP (μ/L) |
|---|---|---|
| 0.0 | 293.8 | 295.4 |
| 1.0 | 303.6 | 352.7 |
| 5.0 | 20.5 | 11.6 |
| 10.0 | 5.6 | 5.1 |

What is claimed is:

1. A method for reducing the disinfection byproduct formation potential of drinking water comprising contacting a raw drinking water sample with a composition comprising a peracid solution to form a peracid treated raw drinking water sample.

2. The method of claim 1, further comprising contacting the peracid treated raw drinking water sample with a disinfecting chemical, thereby disinfecting the drinking water.

3. The method of claim 1, wherein the disinfection byproduct is a trihalomethane or a haloacetic acid.

4. The method as in claim 1, wherein the peracid solution is a performic acid solution, a peracetic acid solution, a perproprionic acid solution, or a combination of a performic acid solution, a peracetic acid solution, or a perproprionic acid solution.

5. The method of claim 4, wherein the peracid solution is a peracetic acid solution.

6. The method of claim 5, where the peracetic acid solution comprises 2-35 wt % peracetic acid and 5-30 wt % hydrogen peroxide.

7. The method of claim 5, wherein the concentration of the peracetic acid in the peracid-treated raw drinking water sample is between 0.5 mg/L and 20 mg/L.

8. The method as in claim 7, wherein the concentration of the peracetic acid in the peracid treated raw drinking water sample is between 1 mg/L and 10 mg/L.

9. The method of claim 7, wherein the peracetic acid concentration is controlled to maintain a concentration between 0.5 mg/L and 20 mg/L.

10. The method of claim 9, wherein the peracetic acid concentration is controlled in a flow-pacing manner, a feed-back control manner wherein the peracetic acid concentration in the raw water is measured by one or more submersible analytical probes, in a feed-forward control manner wherein the total organic content or chemical oxidant demand of the raw water is measured and correlated to the required peracetic acid concentration, or a combination thereof.

11. The method of claim 1, wherein the disinfecting chemical is selected from the group consisting of chlorine, chloramine, chlorine dioxide, and ozone.

12. The method of claim 1, wherein the raw drinking water is contacted with a peracid solution for a time sufficient to reduce the concentration of disinfection byproduct precursors.

13. The method of claim 12, wherein the disinfection byproduct precursors comprise natural organic matter.

14. In method of reducing the level of a disinfection byproduct precursor in a water sample, the method comprising contacting the water sample with a composition comprising a peracid solution for a time sufficient to reduce the level of the disinfection byproduct precursor.

15. The method of claim 14, wherein the disinfection byproduct precursor comprises natural organic matter.

16. The method of claim 14, wherein the water sample comprises water that has not been contacted with a disinfecting chemical.

17. The method of claim 14, wherein the peracid solution is a peracetic acid solution.

18. The method of claim 17, where the peracetic acid solution comprises 2-35 wt % peracetic acid and 5-30 wt % hydrogen peroxide.

19. The method of claim 18, wherein the concentration of the peracetic acid in the peracid-treated raw drinking water sample is between 0.5 mg/L and 20 mg/L.

20. The method as in claim 19, wherein the concentration of the peracetic acid in the peracid treated raw drinking water sample is between 1 mg/L and 10 mg/L.

21. The method of claim 19, wherein the peracetic acid concentration is controlled to maintain a concentration between 0.5 mg/L and 20 mg/L.
